(19) United States
(12) Patent Application Publication
(10) Pub. No.: US 2012/0143213 A1
(43) Pub. Date: Jun. 7, 2012

(54) Surgical Apparatus

(75) Inventor: Mattias Myrman, Tyreso (SE)

(73) Assignee: ELEKTA AB (PUBL), Stockholm (SE)

(21) Appl. No.: 13/390,434

(22) PCT Filed: Aug. 14, 2009
(86) PCT No.: PCT/EP09/05930

§ 371 Date: Feb. 14, 2012

(51) Int. Cl. A61B 19/00

(52) U.S. Cl. 606/130

(57) ABSTRACT

A patient can be taken to the operating theatre where a measurement arm can be used to locate fiducial markers relative to a fixed substrate. As a result, the location of the region of interest is known. An instrument guide, adapted to constrain movement of an instrument thereon, can then be positioned at a suitable location, and its location checked by way of the measurement arm. A computing means connected to the measurement arm so as to receive information as to the location of the tip can be programmed to determine a desired necessary movement of the guide in order to locate the instrument at a desired location relative to the region of interest. The guide can then be moved in a controlled and accurate manner from its initial position to an accurately determined correct position.

### FIELD OF THE INVENTION

The present invention relates to apparatus for use in surgery. The apparatus described addresses particular challenges met in the field of neurosurgery.

### BACKGROUND ART

In the field of neurosurgery, it is often necessary to insert an instrument into the patient's brain tissue in order to perform a surgical step at an internal site. A wide range of examples exist; excess fluid can be aspirated, malignant or malfunctioning regions can be cauterized, or regions can be stimulated using temporary, semi-permanent or permanently implanted probes. In all these (and other) cases, an instrument of some description needs to be inserted so that its operative region—usually the tip—is accurately positioned at a specific location within the cranium.

To allow access, an incision is made in order to reveal the skull, and an aperture is cut in the bone at a suitable location to reveal the soft tissue beneath. It then remains only to align the instrument, which usually has an elongate aspect, so that it can be guided in a straight line towards the specific location. This is a sensitive task that requires a high degree of accuracy in order to avoid collateral damage to other structures within the brain. Generally, an MRI scan is taken prior to surgery, and an insertion route is determined which avoids major blood vessels, internal neurological structures, and the like. Thus, the relevant parameters comprise a 3-dimensional location, and an approach path defined by an elevation angle and an azimuth angle.

The general position of the aperture in the patient's skull is determined by the approach path. However, a high degree of accuracy is needed in positioning the instrument for surgery, in order to conform closely to the chosen path, and also to prevent unintentional movement during surgery. Accordingly, an instrument holder is usually provided which allows the instrument to be mounted on a frame and positioned according to an accurately determined position and approach angle. The longstanding standard in this regard is the Leksell Stereotactic System®, which is a frame that can be positioned in three-dimensions so that a defined centre of arc coincides with the specific location and which can then support an instrument at an adjustable elevation angle and azimuth angle.

To use this (or a similar) system, it must be attached rigidly to the patient and a co-ordinate system must be established which links the position of the specific location with the position of the frame. This then allows the necessary adjustment of the frame to the correct location. To couple the reference system of the patient brain to the reference system of the instrument holder used in surgery, a frame is fixed to the skull and an indicator box containing known geometry fiducials that are visible in MRI is attached to the frame. The frame and box are then included in the MRI scan of the patient, and the frame is thereafter used as the mechanical interface to the instrument holder. In this way, the relative positions of the specific location and the frame can be determined from the scan, given the known geometry of the frame and the indicator box, and the frame itself determines the base position of the instrument holder—whose geometry is also known. A fixed geometric relationship is therefore established between the specific location and the instrument holder, allowing the latter to be positioned with accuracy.

There are a number of problems with this method. The head coils for MR scanning should be positioned as close to the head as possible in order to obtain better resolution images, and thus allow more accurate determination of the specific location. This means that many coils that are in use at present are a very tight fit around the patient's head, and thus the frame may not fit within all available head coils on the market.

Electrically conductive frames in an MR environment can also create heat (through SAR effects) within the fixture, close to the patient's head. This is a potential hazard to the patient. It can be alleviated by limiting the length of the conductive path within the frame, and (especially) avoiding a complete circular conductive path around the patient, but the sheer size of any frame will present difficulties.

Finally, the purpose of the frame is to provide a rigid reference point to which the stereotactic frame and surgical instruments can be repeatedly attached and removed, whilst remaining rigid and accurately positioned. This inevitably requires the use of stiff and resilient materials, which carries a weight penalty. Meanwhile, the frame must be carried by the patient for an extended period of time, resulting in a potential for discomfort.

### SUMMARY OF THE INVENTION

The present invention proposes an alternative approach to surgery, which is able to meet the accuracy needs of neurosurgery. It is of course applicable to other forms of surgery that may require a high positional accuracy, but it is expected that the principal need is within the neurosurgical field.

According to the invention, the patient is scanned, either via MRI or by such other method as is desired, with one or more fiducial markers in place. A plurality of markers is preferred, such as at least three, so that the markers provide an unambiguous three-dimensional orientation. Typically, four or five markers will be used in order to provide a degree of redundancy and highlight any measurement errors. From this scan, the location of a region of interest within the patient can be determined, relative to the fiducial marker(s).

The patient can then be taken to the operating theatre, perhaps with a delay after scanning to allow for efficient scheduling or for surgical planning to take place. In the operating theatre, a measurement arm can be used to locate the fiducial markers relative to a fixed substrate such as a head fixation device, which then permits the frame of reference of the scan to be registered with the frame of reference of the operating theatre. As a result of this, the location of the region of interest relative to a substrate is known.

An instrument guide, adapted to constrain movement of an instrument thereon, can then be put at a suitable approximate location, and its location measured by way of the measurement arm. A suitable computing means, connected to the measurement arm so as to receive information as to the location of the tip, can be programmed to determine a desired necessary movement of the guide in order to locate the instrument accurately at a desired location relative to the region of interest. The guide can then be moved in a controlled and accurate manner from its initial position to an accurately determined correct position. A surgical instrument located on the guide will then be accurately positioned for surgery. Typically, in a neurosurgery application, this position will be one in which the instrument is ready to translate along its length, penetrating the brain tissue, until its tip or other operative portion is correctly located within the patient. This movement can, likewise, be tracked by the measurement arm if desired.

In this way, the invention permits MRI or other scans to be taken without bulky or potentially hazardous head frames needing to be present. Nevertheless, a high degree of accuracy can still be maintained in the operating theatre.

A measurement arm is an adjustable articulated arm ending with a probe having a tip, and which is adapted to report a location of the tip and (usually) a direction of the probe. Typically, each articulation of the arm includes a calibrated encoder that reports the instantaneous angle of that articulation. As a result of this, given the known geometry and dimensions of the arm, an instantaneous position of the tip can be calculated. Such arms are commercially available from Faro Technologies Inc of Lake Mary, Fla.

The present invention therefore provides, in its first aspect, surgical apparatus comprising an adjustable articulated arm having a tip, and being adapted to report a location of the tip, a guide adapted to constrain movement of an instrument thereon, a surgical instrument located on the guide, a computing means connected to the articulated arm to receive information as to the location of the tip, the computing means being programmed to determine a desired necessary movement of the guide in order to locate the instrument at a desired location.

The instrument is preferably supported by the guide, to or in which it can be clamped. Such instruments are usually elongate in nature, and the guide is preferably adapted to constrain translational movement of the instrument to translation along its elongate axis only.

A particular advantage of the invention is that it permits the straightforward provision and control of a servomechanical adjustment means for the guide. This can comprise one or more servo-controlled motors for adjusting the guide, under the control of the computing means. Ideally, once the guide has been manipulated into an approximate position by hand, and its actual location determined by use of the measurement arm, the computing means can then determine a correction that needs to be made to the guide position and control the servomechanical adjustment means to move the guide by the required correction (preferably under a closed-loop control system) and thereby position the guide accurately at the correct location. The initial approximate position of the guide can be determined by using the measurement arm to report the position of specific (known) points on or associated with the guide, from which the guide position can be calculated. Alternatively, or in addition, the probe of the measurement arm can be mounted in or with the guide in a known relationship thereto, and the position and orientation of the probe can then be used to determine the position of the guide. The latter approach is particularly suited to a closed-loop control system, as the probe can be left in place while the guide position is corrected from its initial approximate position to its intended accurate position.

The guide preferably comprises at least two supports for the instrument, each support being moveable independently. This allows the orientation of the guide to be adjusted automatically in all the necessary degrees of freedom, i.e. the required translational axes, together with azimuth and elevation angles, and possibly also depth (relative to the patient).

The instrument is usually one adapted to perform at least one of aspiration, injection, cauterization, electrical stimulation, and placement of items such as electrodes, containers, controlled-release formulations, and the like. However, its exact function is not germane to the present invention, which is primarily concerned with the accurate positioning of the instrument.

In a second aspect, the present invention also provides a method of guiding an instrument to a location relative to a feature within a physiology, comprising the steps of performing a scan of the physiology to determine the location of the feature within the physiology relative to at least one external feature of the physiology, providing an adjustable articulated arm having a tip and adapted to report a location of the tip, employing the arm to determine a location of the or each external feature, employing the arm to determine a location of the instrument, and with the thus acquired knowledge of the location of the instrument relative to the location of the feature, guiding the instrument to the feature.

The external feature is preferably a marker attached to the physiology such as the fiducial markers discussed above.

Typically, the physiology will be a human anatomy.

Other preferable features of this aspect will be as those which were discussed above in relation to the first aspect.

### BRIEF DESCRIPTION OF THE DRAWINGS

An embodiment of the present invention will now be described by way of example, with reference to the accompanying figures in which;

FIG. 1 shows the principal items of surgical equipment;

FIG. 2 shows the measurement arm;

FIG. 3 shows a patient prior to scanning with a plurality of implanted fiducials;

FIG. 4 shows the patient being scanned;

FIG. 5 shows the scan result;

FIGS. 6 to 9 show sequential steps in preparing the patient for surgery;

FIG. 10 shows the registration of the fiducials with the measurement arm;

FIG. 11 shows the servomechanical adjustment device;

FIG. 12 shows the servomechanical adjustment device fitted and being registered;

FIG. 13 shows the instrument guides in an initial coarse location, ready to be positioned;

FIG. 14 shows the instrument guides in their accurate position;

FIG. 15 shows the surgical instrument ready to be inserted;

FIG. 16 shows the instrument inserted and being verified in situ;

FIG. 17 shows a second embodiment of the invention;

FIG. 18 shows the registration of the xy unit in the second embodiment;

FIGS. 19 and 20 show the adjustment of the xy unit;

FIG. 21 shows a head frame of a third embodiment;

FIG. 22 shows the registration of the head frame;

FIG. 23 shows the MR scanning step of a fourth embodiment;

FIG. 24 shows the head frame of the fourth embodiment;

FIG. 25 shows the locator unit for the frame;

FIG. 26 shows the arrangement for CT scanning;

FIG. 27 shows the patient in place for surgery; and

FIG. 28 shows the registration of the head frame.

### DETAILED DESCRIPTION OF THE EMBODIMENTS

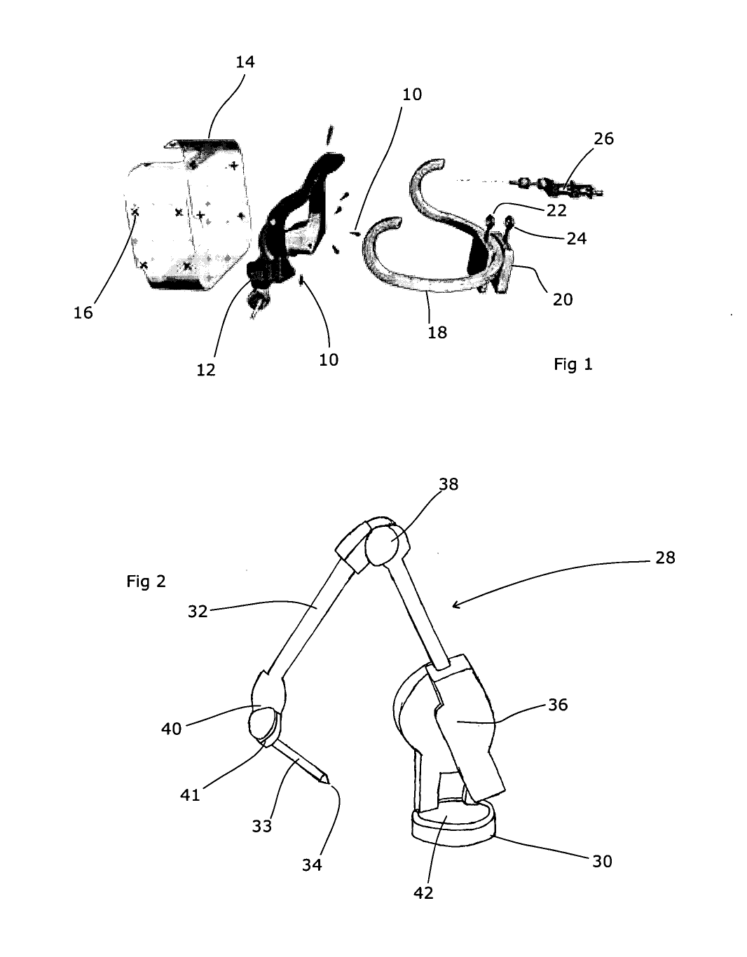

FIG. 1 illustrates the principal parts of the first embodiment of the invention. A plurality of fiducial markers 10 are provided, and are attachable to a patient such as by screwing into a cranium. A head fixation 12 is provided, which can be used to restrain a part of the patient such as the head, and provide an attachment point for other devices (as will be described). This is connected to the table at a lower extremity of the device and contains suitable fixings for securing to the head in order to immobilise it. An x-ray indicator box 14 is also provided, which comprises a tubular structure which can be attached to the head fixation base 12 around the patient. A number of indicia 16 are formed in the indicator box and are visible in MRI and/or CT scanning. Many other forms of indicator boxes could be employed, preferably of a less obtrusive nature so as to permit simultaneous fitting of other devices.

An instrument arm 18 can be attached to the head fixation base in a rigid manner. This carries an adjustable fine adjustment unit 20. Both the angle of attachment of the instrument holder arm to the head fixation base and the azimuthal position of the fine adjustment unit 20 along the instrument holder arm are adjustable. In this way, an initial rough position for the fine adjustment unit can be achieved manually.

The fine adjustment unit 20 contains a forward support 22 and a rear support 24 for a surgical instrument 26, both of which extend from the chassis 25 and end in a ring structure able to accept corresponding external structures of the surgical instrument 26. Both the forward support 22 and the rear support 24 are adjustable in two (x,y) directions, each direction being within the plane that includes their longitudinal extent and the ring structure. A further motor permits movement of the two supports 22, 24 together in a third, perpendicular, direction (z). This will be explained further in relation to later figures.

FIG. 2 shows the measurement arm 28. This includes a base 30 which can be permanently or semi-permanently fixed to a suitable substrate, such as the patient table, by clamping or by other suitable means. An arm 32 ending in a probe 33 having a tip 34 finished to a sharp point then extends from the base 30 via (in this case) a total of four articulations 36, 38, 40, 41. Each articulation includes a calibrated encoder to record the angle(s) subtended at that articulation. In addition, a rotatable section 42 is included within the base 30, allowing the arm to rotate freely and thereby permitting the tip to reach substantially any point. The encoders in each articulation record the angle or angles through which that articulation has been deflected, and from this and prior knowledge of the lengths between each articulation it is possible to determine accurately the position of the tip relative to the base. Typically, such arms are provided together with dedicated software that performs this calculation in real time. Whilst it is not possible to determine an absolute position from such a device, as the position of the base 30 is in principle unknown, multiple readings will reveal the position of one item relative to other items.

The arm illustrated in FIG. 2 thus has a total of five degrees of freedom, i.e. the four articulations and the rotatable base. Other designs of arm are possible, and may have more or (possibly) fewer degrees of freedom. Generally, the exact geometry of the arm may be varied to suit the particular geometry of the other equipment on which the arm is to be mounted and whose position is to be determined.

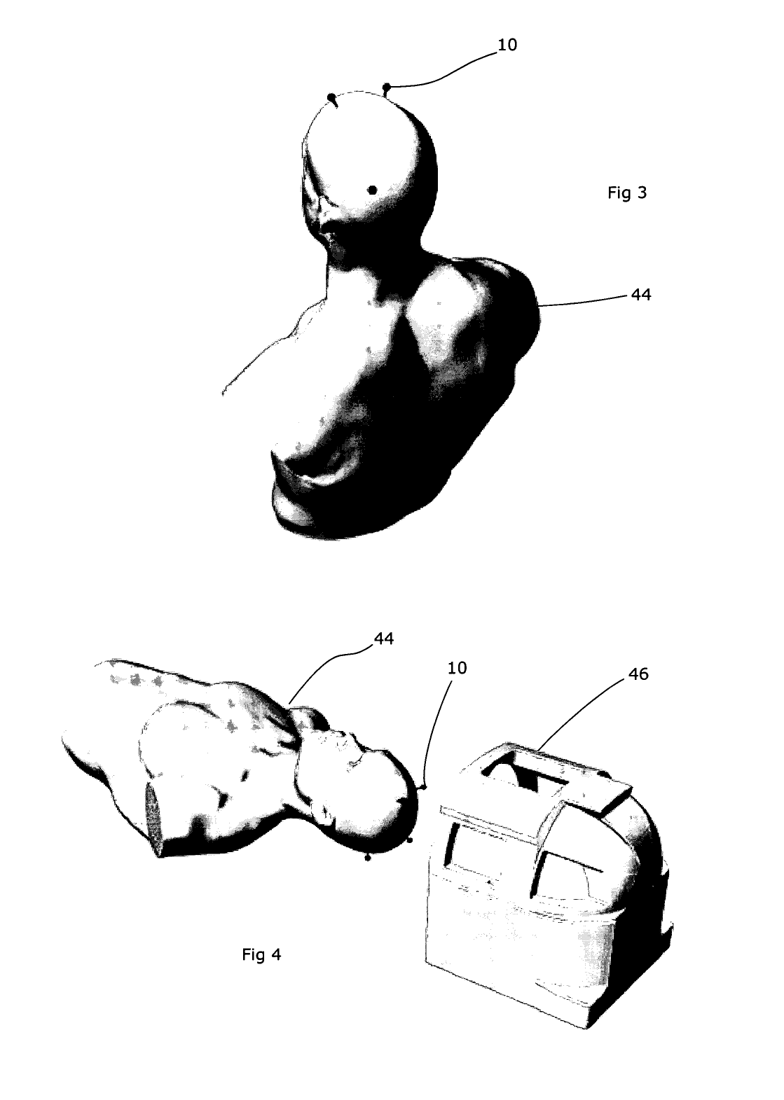

FIG. 3 shows a patient 44 being prepared for surgery. A plurality of (in this case 4) fiducial markers 10 are attached to the skull of the patient 44 in a generally known manner. Typically, this involves creating an incision through the overlying tissue (under a local anaesthetic) followed by screwing the fiducials into the underlying cranium. A range of fiducial markers are available. Each typically includes a self-tapping screw threaded portion, to allow fixation to the skull or other bone. At the free end, a spherical tip is often provided.

FIG. 4 shows the patient being scanned by MRI. The patient 44, with fiducial markers 10, is placed within an MRI head coil 46 and scanned. The scan will show internal features of the patient's cranial physiology, in combination with the fiducial markers 10.

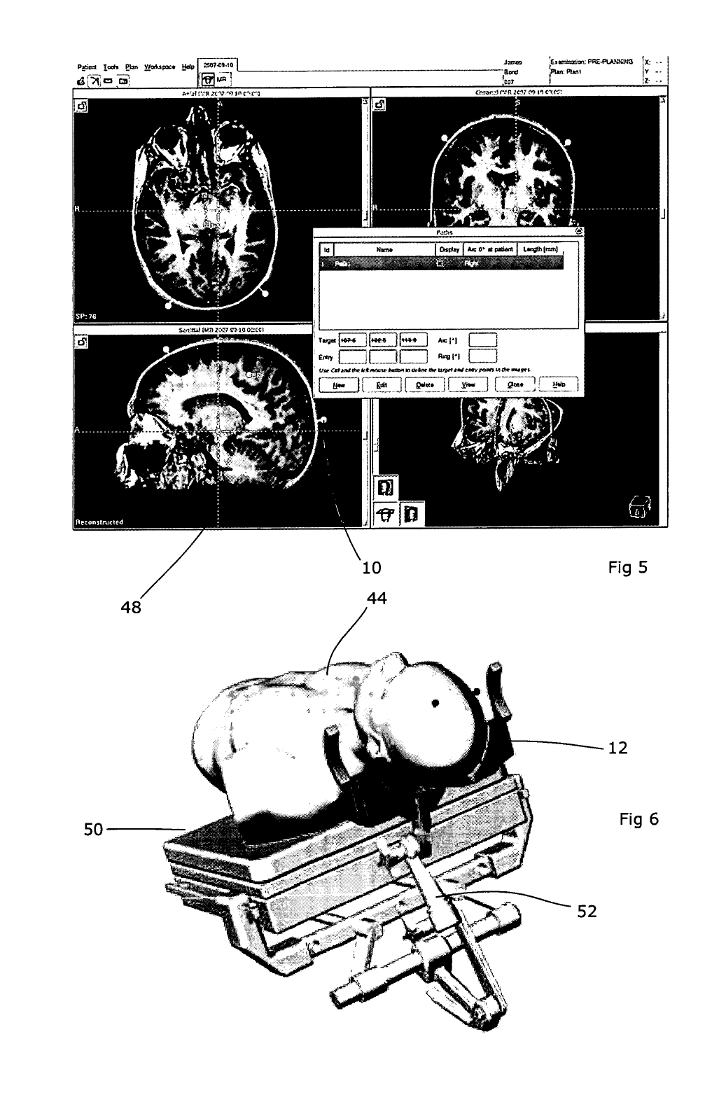

FIG. 5 shows a typical output 48, from which the location of the regions of interest relative to the fiducial markers 10 can be ascertained. This can be used by a surgeon to determine an acceptable route to the region of interest, avoiding sensitive structures, from which the necessary azimuthal and elevation angles leading to the region of interest can be determined. The images also show the locations of the fiducial markers 10 relative to the region of interest, and allow subsequent use of the location of the markers to determine the location of the region of interest.

After a period of time to allow for efficient scheduling and/or surgical planning, if necessary, the patient can then be prepared for surgery in the operating theatre. During the intervening period, the patient need only cope with the weight and bulk of the fiducial markers, rather than an entire head frame. The patient 44 is placed on a patient table 50 and their head allowed to rest within the lower section of the head support 12. This is supported rigidly at 52 so that its position remains stable during the procedure.

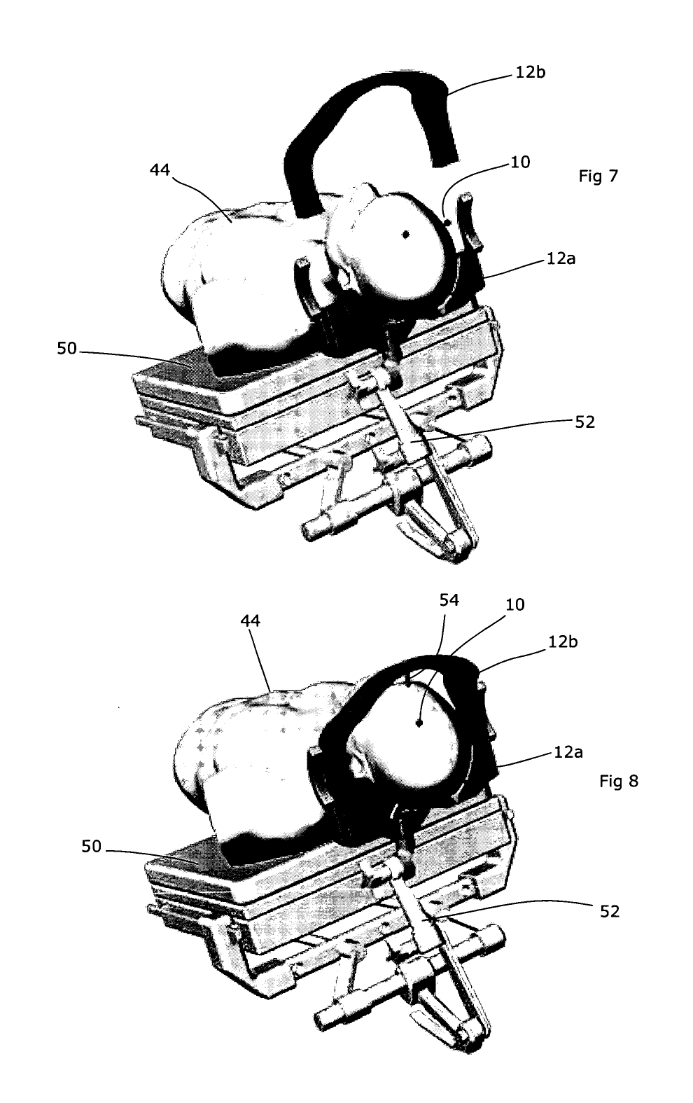

As shown in FIG. 7, an upper section of the head fixation 12b of the head fixation 12 is then fixed in place over the lower section 12a and the head of the patient 44 is restrained by suitable clamps 54.

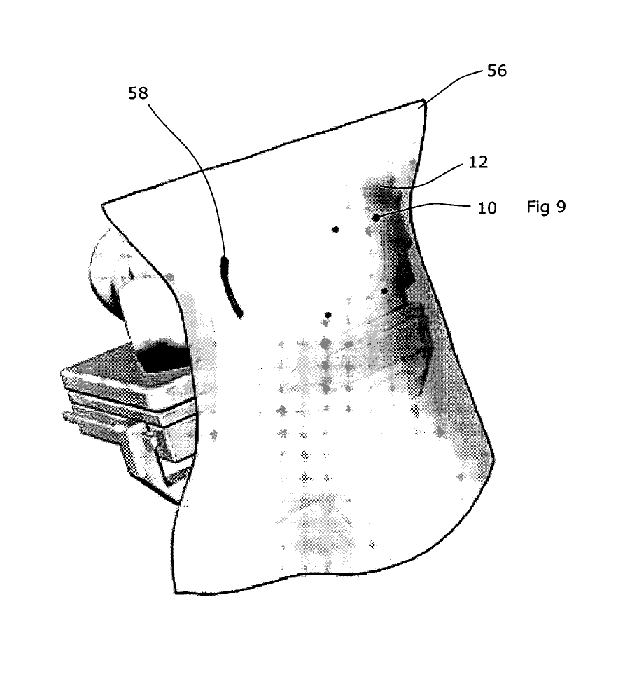

A drape 56 can then be added, in accordance with good operating theatre practice. The fiducials 10 and the attachment points 58 on the head fixation frame 12 should be allowed to project through the drape. This is shown in FIG. 9.

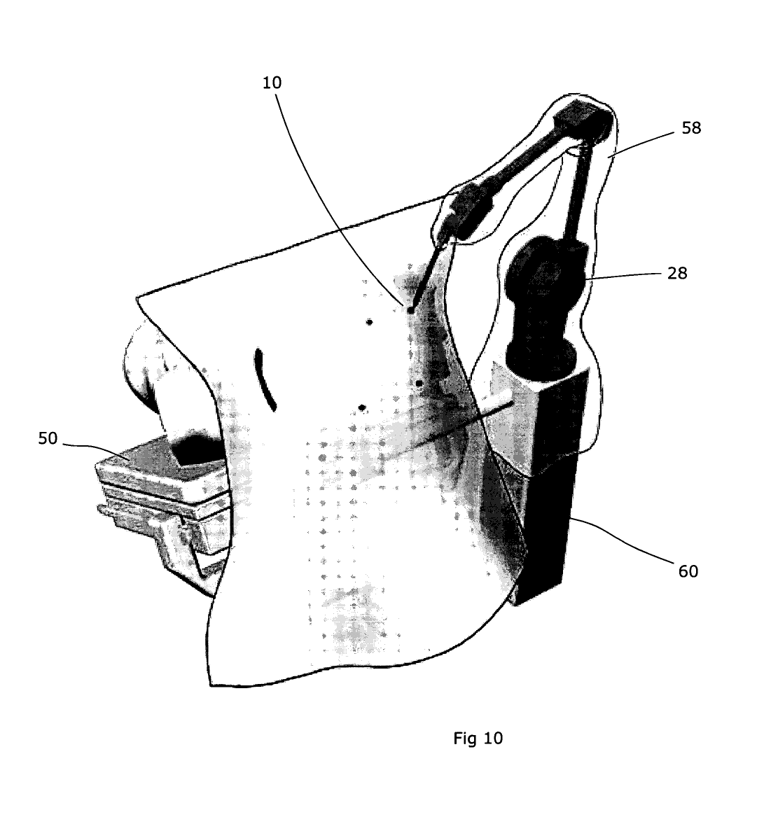

As shown in FIG. 10, the measurement arm 28 together with suitable sterile draping 58 of its own can then be attached rigidly to a suitable support 60 that is fixed rigidly relative to the head frame 12, and used to ascertain a position of each of the skull fiducials 10. A pointed tip 34 to the measuring arm 28 can be placed in a tapering recess formed within the skull fiducials 10 to provide unambiguous location information. If the recess is coincident with the centre of the spherical head of the fiducial then this will allow the measurement arm 28 to ascertain a position of the centres of the spherical formations of the skull fiducials 10 to a high degree of accuracy without the need for further computation. Alternatively, the probe 33 of the measurement arm 28 could be provided with a fixed or temporary tip having an internal cone-shaped recess into which a spherical head of the fiducial 10 could fit, again providing an unambiguous location.

An incision can then be made in the patient's scalp followed by a burr hole formed in the skull in the usual manner, to expose the brain tissue and allow access. The incision is usually a circular aperture of approximately 15 mm diameter, created with a suitable cutting tool. The aperture can be centred approximately on the intended insertion point of the instrument, and its size allows for positional tolerances and last-minute adjustments to the exact insertion point.

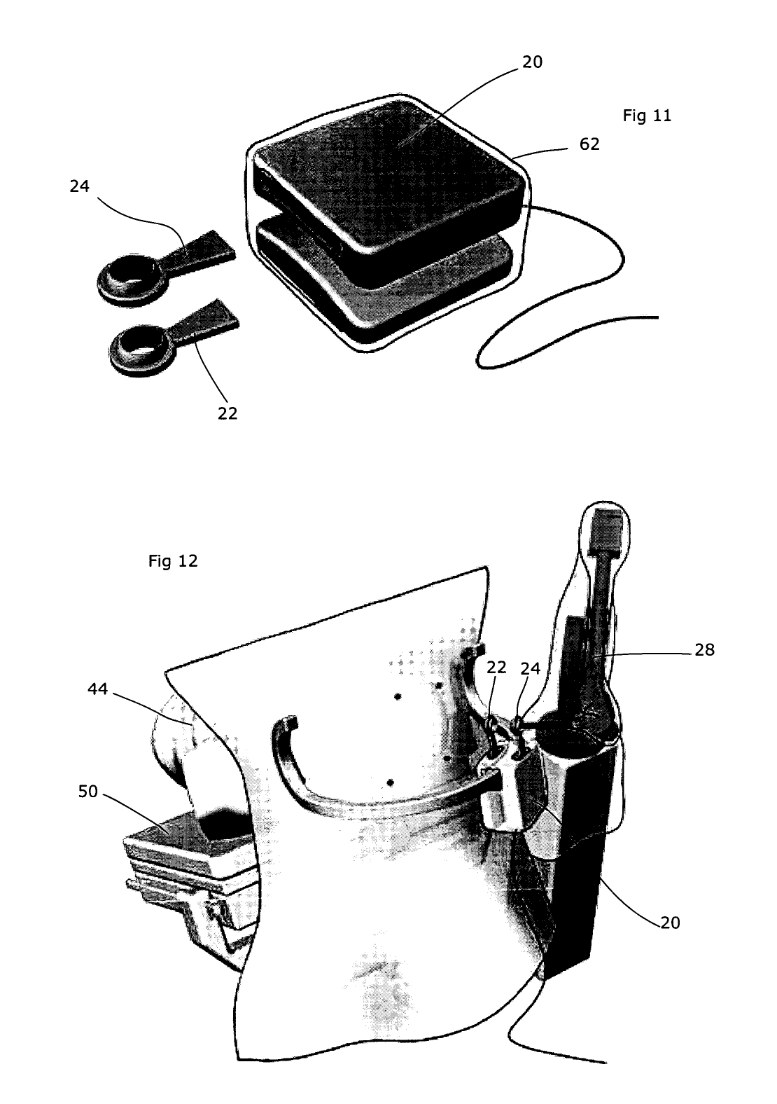

The fine adjustment unit 20 is then prepared. A suitable form of draping 62 is provided around the operative parts of the fine adjustment unit 20, and the front and rear supports 22, 24 project out of the draping 62. These are removable so as to allow for sterilisation. The instrument holder arm 18 is attached to the head fixation unit 12 and the fine adjustment unit 20 is mounted to the instrument holder arm 18 in an approximately correct position relative to the patient. The measurement arm 28 is then (as shown in FIG. 12) used to determine the actual position of the front and rear adjustment arms 22, 24. Suitable tapered formations can be provided within the arms for this purpose, or other topological features of the arms can be used to provide this necessary level of definition.

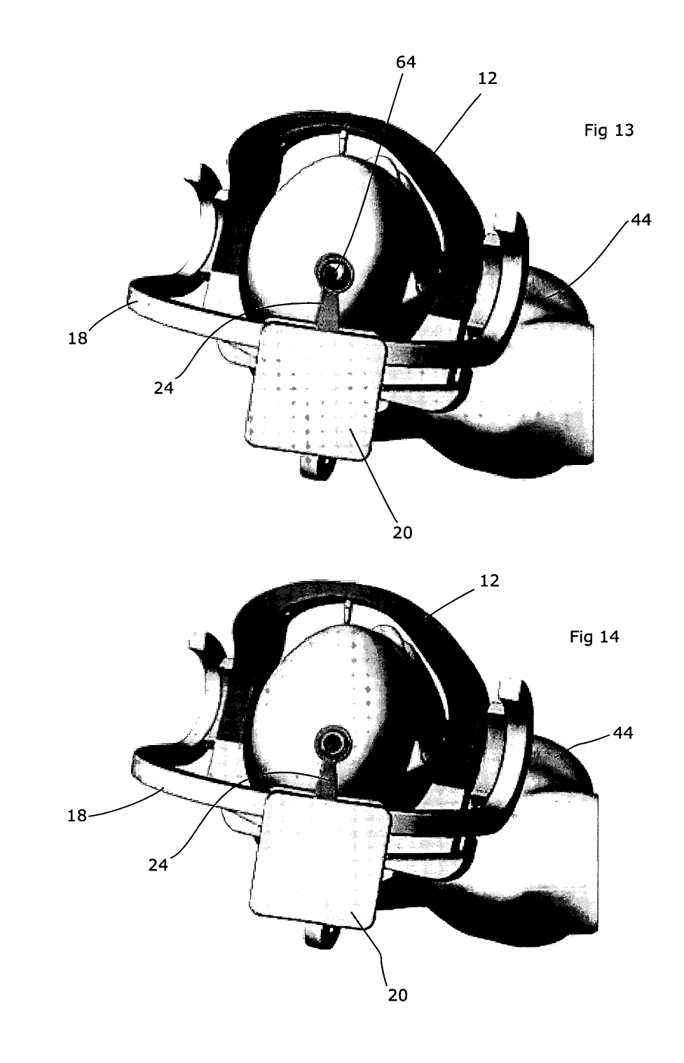

This then produces the arrangement shown in FIG. 13 in which (with draping omitted for clarity) the front and rear support arms 22, 24 are in an approximate position relative to an incision 64 in the patient's cranium. This approximate position represents the position which the items are initially placed by the surgeon. However, as a result of the registration of the skull fiducials 10 by the measurement arm 28, and the registration of the support arms 22, 24, and the MRI scan correlating the patient's internal structure with the skull fiducials 10, the degree of error between the current position of the support arm 22, 24 and their ideal position can be calculated by straightforward geometrical calculations. This error for each arm can then be fed to the fine adjustment unit 20 so that each arm is adjusted in the x and y directions, which will define a plane roughly tangential to the local surface of the patient's head. This will place them in the position shown in FIG. 14 in which they are perfectly aligned with the desired direction of entry into the patient's cranium.

Two motors will thus be provided within the fine adjustment unit 20 for each of the support arms 22, 24 in order to move each of them independently in both the x and y directions. A fifth motor is also provided, able to move both arms 22, 24 bodily relative to a chassis within the fine adjustment unit 20 in a third (z) direction perpendicular to the x and y directions. This z direction will therefore be towards and away from the patient, and adjustment in this direction will allow the positions of the support arms 22, 24 to be adjusted to position them (and the instrument guide) at the correct distance from the region of interest within the patient. Most instruments are generally about 200 mm long, so that distance will usually be chosen so as to place the relevant part of the instrument or instrument guide 190 mm from the region of interest.

The fine adjustment unit 20 has a movement range of 40 mm for each arm 22, 24 in the x and y directions, and a range of 20 mm in the z direction. With a spacing between the arms of 50 mm, this allows a good range of positions and angles for the instrument guide.

Instrument guides often include an adjustable “stop” for the instrument, allowing the user to set a reference distance so that the instrument is inserted to a desired depth (relative to a reference position). An alternative to a z adjustment function would therefore be to provide a guide with a manually adjustable zero position or reference position for the depth (z), the nominal position of which is related to the length of the instrument.

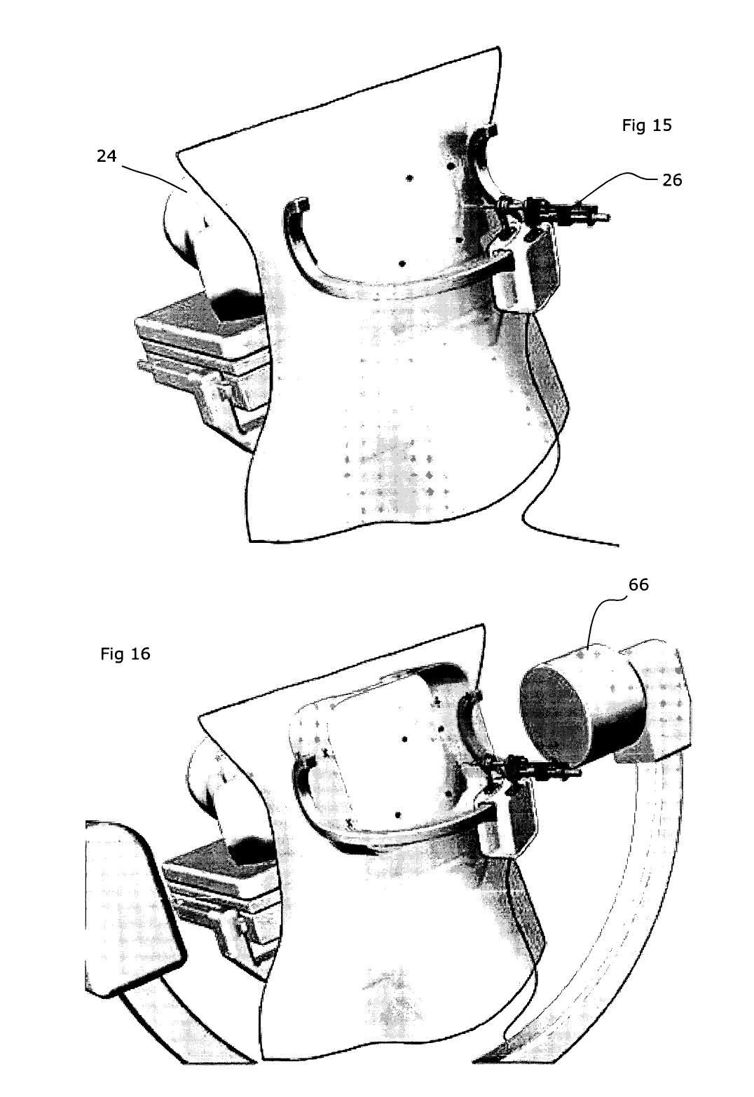

As shown in FIG. 15, an instrument holder or an instrument 26 can then be fitted to the support arms 22, 24 and inserted into the skull of the patient 44. Such instrument holders guide an instrument along a straight line by a measured distance in order to guide the tip of the instrument to the intended location. Optionally, as shown in FIG. 16, once the instrument 26 is in position, an x-ray unit 66 can be brought into the operating theatre to prepare a swift x-ray image of the patient with the instrument inserted in order to confirm that the instrument is indeed inserted to the correct position and the correct depth.

Alternatively, or in addition, software could keep track of the tip of the instrument during surgery, i.e. as it was inserted via the instrument guide, and create a virtual image on top of the MRI image of a needle progressing through towards the target. This would enable the surgeon to see where the tip is located in real time.

Thus, the first embodiment of the invention allows surgery to be conducted in an accurate manner, without having to expose the patient to MR scanning whilst fitted with a large and bulky frame of a conductive material. Instead, a number of small fiducial markers 10 can be employed which are compatible with MR imaging and which are not overly intrusive during the period between scanning and surgery.

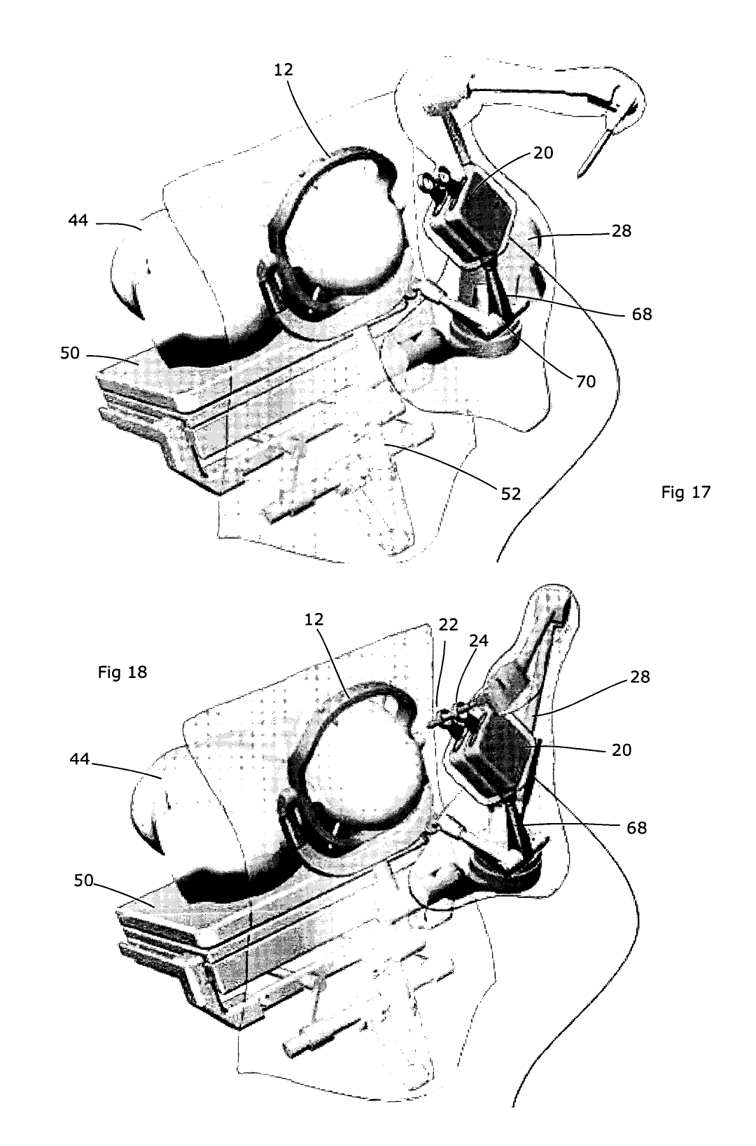

There are various manners in which this first embodiment can be varied. The first of these is shown in the second embodiment, illustrated with reference to FIGS. 17-20. In this embodiment, as shown in FIG. 17, at the stage when the fine adjustment unit is introduced during surgery (equivalent to FIG. 12 in the first embodiment), instead of being mounted on the support 18 it is instead mounted on a sterilised 3D arm 68. 3D arms are employed in other fields and consist of an elongate arm having an articulation 70, roughly at the mid-point of the arm, and articulated attachment interfaces at either end of the arm 68. The articulation 70 includes a lever or other actuating handle which can be set in one of two positions. In a first position, the articulation 70 is free to rotate and the interfaces at either end of 3D arm 68 are free to articulate angularly in two degrees of freedom. In a second position of the operating lever, the 3D arm 68 is locked into the position it held while the lever was operated, and no further movement of the articulation 70 and the engagement portions is permitted while it remains locked. Generally, the articulation 70 allows the two parts of the arm to hinge into a V-formation thereby adjusting the absolute distance between the two engagement formations at either end of 3D arm 68. This, together with the articulation of the interface portions in two degrees of freedom allows the 3D arm 68 to position the fine adjustment unit 20 in essentially any orientation at essentially any location relative to the patient table 50, within obvious limitations of reach.

Accordingly, the surgeon attaches one engagement portion of the 3D arm 68 to the head frame 12, attaches the fine adjustment unit 20 to the other interface portion at the other end of the 3D arm 68, and operates the operating lever so as to release the 3D arm 68 and permit movement of its various articulations. The fine adjustment unit 20 is then placed in an approximately correct position, as shown in FIG. 17, and the operating lever is re-adjusted so as to lock the 3D arm 68 in its instantaneous position.

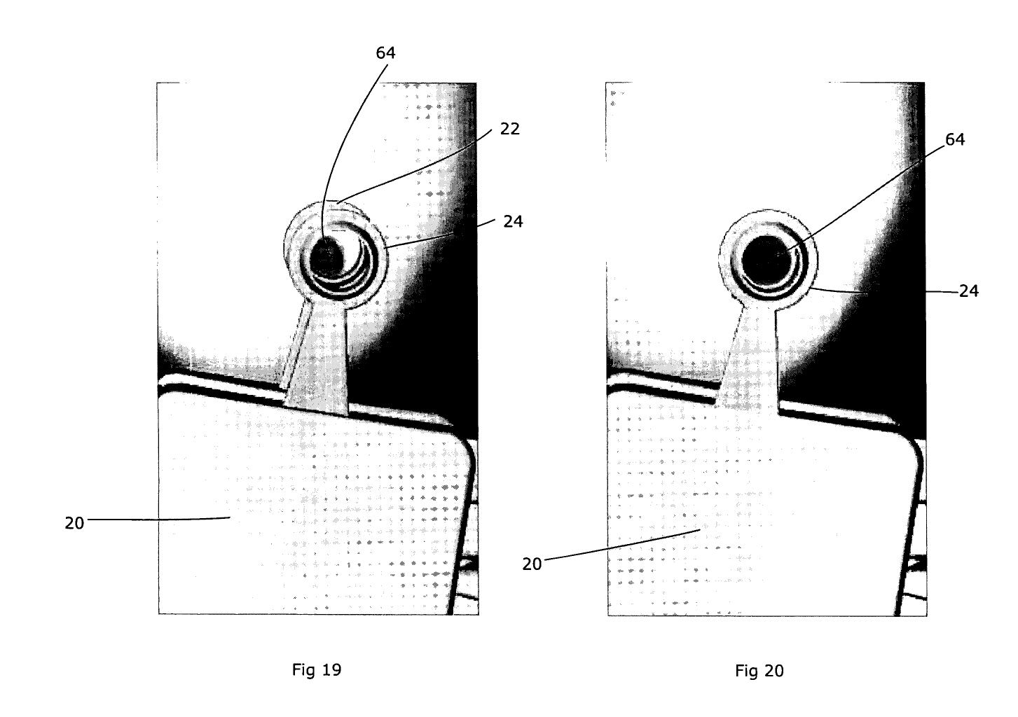

As shown in FIG. 18, the measurement arm 28 is then brought to the fine adjustment unit 20 in order to register the instantaneous position of the fine adjustment unit 20 and, particularly, the front and rear instrument holders 22, 24. Earlier in the procedure, as with the first embodiment, the measurement arm 28 will have been used to register the position of fiducial markers in the patient's skull, meaning that the orientation of the instruments was 22, 24 relative to the desired location within the patient's physiology can be calculated as before. This allows the instrument supports 22, 24 to be adjusted relative to the incision 64 from their initial approximate position shown in FIG. 19 to their accurate position shown in FIG. 20.

FIG. 18 shows the probe 33 of the measurement arm 28 fitted into the support arms 22, 24. Suitable formations can be provided on the support arms 22, 24 and/or the probe 33 to allow the probe 33 to be fitted in a unique position relative to the support arms 22, 24. This allows the software that reports the position of the measurement arm 28 to report the positions of both support arms 22,2 4 simultaneously, and also to report the vector (direction) of the support arms 22,23.

Thus, the second embodiment employs a 3D arm 68 instead of the support 18 in order to carry the fine adjustment unit 20 and allow a greater degree of freedom and flexibility to the surgeon in the operating theatre. In addition, for complex surgery in which multiple instruments are to be inserted at multiple locations on the patient, a corresponding number of 3D arms 68 can be fitted to the frame 12 in order to support a corresponding number of xy-units 20. Each can then be registered individually as shown in FIG. 18, and adjusted so that their respective instrument guides are appropriately aligned. This caters for surgical procedures in which it is necessary (for example) to stimulate or operate upon regions in both hemispheres of the brain. In such operations, there will accordingly be a further 3D arm 68 and fine adjustment unit 20 on the opposite side to that illustrated in FIG. 18.

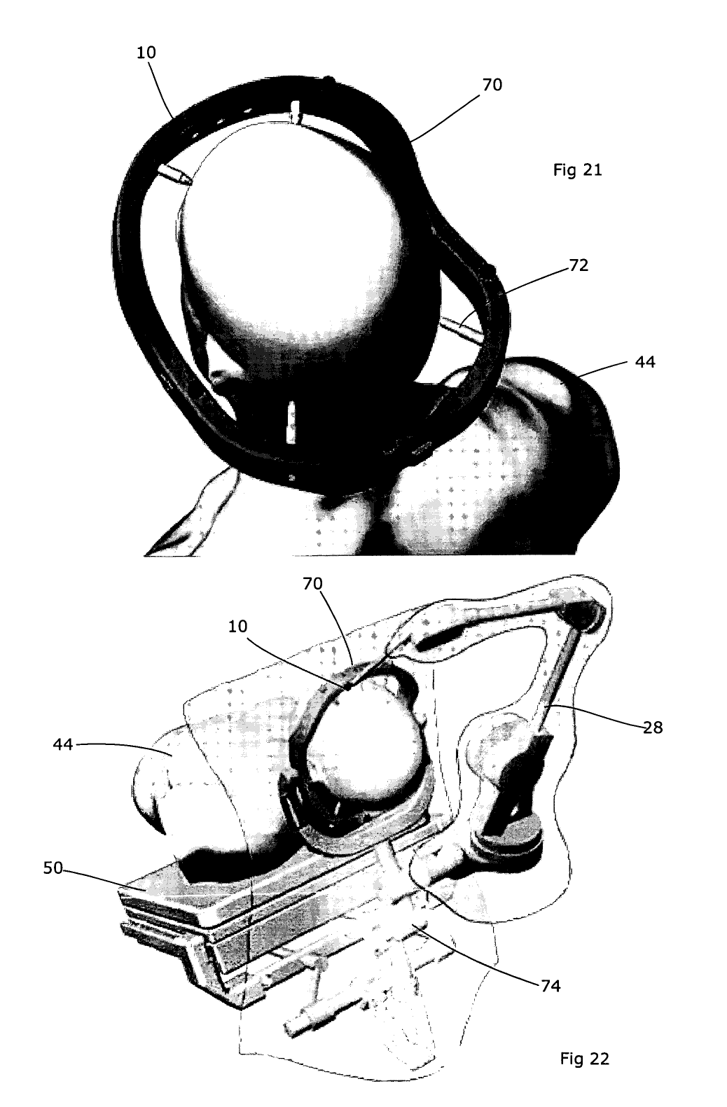

A third embodiment is illustrated with respect to FIGS. 21 and 22. A lightweight non-conductive and non-magnetic head frame 20 is provided which is fixed relative to the head of the patient 44 using pins 72. The inert frame 70, of a material such as a ceramic, polymeric or composite material, presents no risk of heating during MR imaging and is therefore advantageous relative to some existing arrangements. The frame 70 can be provided with multiple fiducial markers 10 of the same type as previously discussed. It can be mounted on the patients as shown in FIG. 21 prior to imaging and surgery and will present little discomfort to the patient in the period between imaging and surgery due to its potential light weight.

Once in theatre for surgery, as shown in FIG. 22, the patient 44 can recline on the operating theatre 50 and the frame 70 can be supported by a suitable adjustment mechanism 74, as before. The measurement arm 28 can then be applied to register the positions of the fiducial markers 10 fixed to the frame 70. As these fiducial markers will also be visible in the MR imaging, this provides the necessary registration of the MR image to the frame of reference that exists within the operating theatre. The surgical instrument can then be supported and guided into position as described with reference to either the first or second embodiments.

It should be noted that such a head frame 70 is unlikely to be completely rigid, as this would be incompatible with the requirements that it be lightweight, non-conductive and non-magnetic. Thus, as it is fitted and the pins 72 are tightened, the frame 70 is likely to become distorted as compared to its original (unstressed) shape. However, according to the present invention this is not problematic, as the fiducial markers 10 are attached to the frame 70 which is left undisturbed between the scanning step and the subsequent operation. Thus, the relative position of the fiducial markers 10 and the patient's internal structures remains the same notwithstanding any initial distortion of the frame 70. It is particularly preferable for the support structures provided for the frame in the scanner and in the operating room to be identical, and therefore any additional distortion which takes place as the patient reclines and weight is transferred to the frame 70 is identical in both locations. Again, therefore, such distortion is non-problematic as the spatial relationship between the fiducial markers 10 and the patient remains identical in both locations.

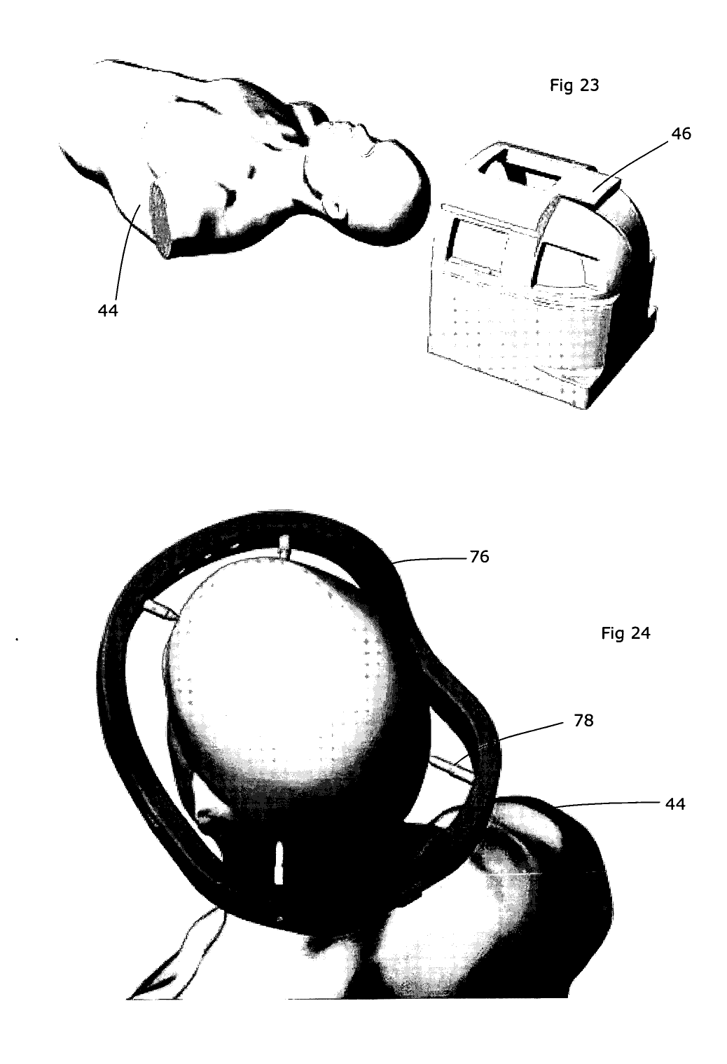

Finally, a fourth embodiment is described with reference to FIGS. 23-28. The patient 44, without any form of frame or implanted fiducial reference, is scanned using MR imaging within a head coil 46. In the absence of any implanted items, discomfort to the patient is of course completely eliminated and the narrowest head frame 46 can be used as desired by the surgical team.

After scanning, a head frame 76 is provided, and is again retained by suitable adjustable pins 78. This is fitted around the head of the patient 44 as shown in FIG. 24 and provides a physical frame of reference around the patient for use in the operating theatre.

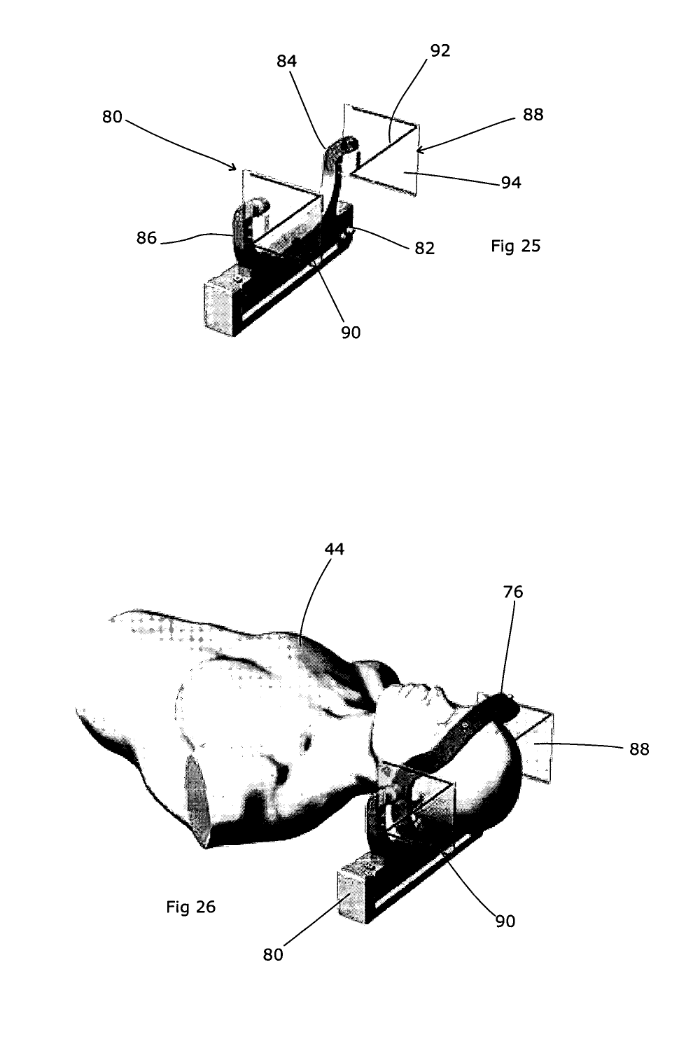

In order to register this physical frame of reference with the frame of reference of the MR image, a CT-compatible (computed tomography) imaging reference 80 (FIG. 25) is fitted to the frame 76 as shown in FIG. 26. This frame of reference 80 consists of a support 82 which can be attached to the head frame 76, from which extend a pair of arms 84, 86. These each carry a semi-transparent reference marker 88, 90, in which there is an x-ray-opaque calibration mark 92 embedded within an otherwise x-ray-transparent substrate 94 (alternatively, an x-ray-transparent calibration mark 92 could be embedded within an otherwise x-ray-opaque substrate). The patient 44, carrying the head frame 76 and the calibration markers 88, 90, is then scanned using known CT apparatus, which will create an image of the internal physiology of the patient 44 together with the calibration markers 88, 90. These two sets of images share common information regarding the patient's internal physiology, and can thus be co-registered as described (for example) in our earlier application WO2006/066791, the contents of which are hereby incorporated by reference and to which the skilled reader should refer for a full understanding of the present invention. As a result, a composite information set is obtained in which the patient's physiology is imaged with the accuracy and sensitivity available using MR scanning, together with the frame of reference information obtainable from the CT scan. This permits the desired regions of interest within the physiology of the patient 44 to be located relative to the head frame 76 despite the fact that that was not present in the MR scan.

The patient can then be taken to the operating theatre and placed on the operating table 50 as shown in FIG. 27. The head frame 76 can be supported by a suitable mechanism 96, as before. After a drape 98 has been added in the usual manner, the measurement arm 28 can be fitted to the mechanism 96 and can be used to register the current position of the head frame 76 or (as desired) a support for the head frame 96 that is in a known geometric relationship with the head frame 76. In this way, the position of the desired feature within the physiology of the patient 44 is then known relative to the frame of reference at the operating theatre. A surgical instrument can then be positioned approximately as described in any of the preceding embodiments, registered relative to the frame of reference at the operating theatre, adjusted into an accurate position, and used for surgery as previously described.

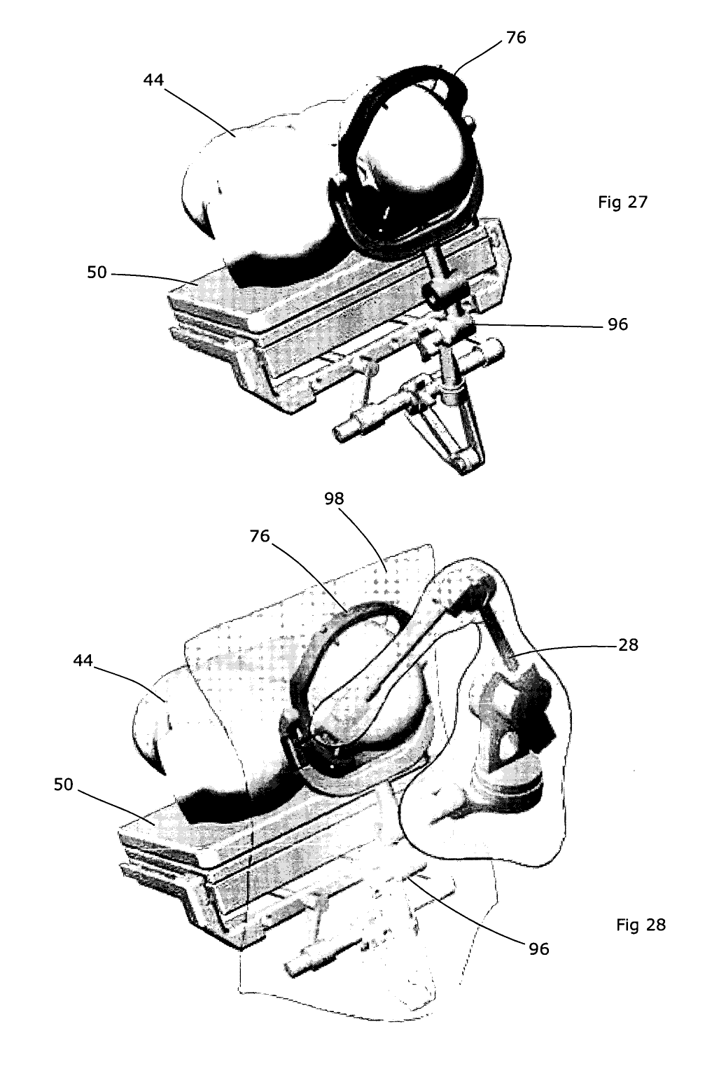

In the operating theatre, the support mechanism 96 supports the head frame 76 in a manner that is geometrically identical to that of the support 82 used during the scanning phase. Thus, any distortion of the head frame 76 that takes place when weight is placed on it is identical in both locations, and the necessary spatial relationships are preserved. FIG. 28 shows registration of part of the support mechanism 96, whose spatial relationship with the CT imaging reference 80 is known as a result of this identicality.

In all of the above embodiments, the support 20 for the surgical instrument is positioned approximately relative to the patient 44 and then guided into an accurate location via a suitable computing means and actuator. That computing means draws on information acquired via the articulated arm 28 to determine or infer the position of the desired location within the patient 44 relative to the current approximate position of the surgical instrument or its guides. There is in each case an unbroken link between the instrument (or its guides) and the desired location, running (for example) from the instrument or guide to the fiducial markers via the measurement arm 28 and from the fiducial markers to the desired location via an MR image. Corresponding relationships exist for each of the embodiments.

An especially preferred embodiment of the invention will include the features of all of the above embodiments, making them available as alternatives to be chosen between by the surgeon. Different surgeons often have different preferences, so a kit from which the preferred elements can be selected is likely to be the most preferably product.

Thus, according to the present invention, a reliable method and apparatus can be provided for guiding an instrument to a specific defined location within a patient, such as within the patient's cranium. This is done in a straight forward and easily understandable manner, which does not require large conductive items to be included with the patient in the MRI scanner, and which can also exclude large bulky items from the scanner.

It will of course be understood that many variations may be made to the above-described embodiment without departing from the scope of the present invention.

1. Surgical apparatus comprising:
an adjustable articulated arm having a tip, and being adapted to report a location of the tip;
a guide adapted to constrain movement of an instrument thereon;
a surgical instrument located on the guide;
a computing means connected to the articulated arm to receive information as to the location of the tip;
the computing means being programmed to determine a desired necessary movement of the guide in order to locate the instrument at a desired location.

2. Apparatus according to claim 1 further comprising at least one marker attachable to a physiology.

3. Apparatus according to claim 2, in which there at least three markers.

4. Apparatus according to claim 1 in which the instrument is supported by the guide.

5. Apparatus according to claim 1, in which the instrument is elongate and the guide is adapted to constrain translational movement of the instrument to translation along its elongate axis.

6. Apparatus according to claim 1, further comprising servomechanical adjustment means for the guide, controlled by the computing means.

7. Apparatus according to claim 1, in which the guide comprises at least two supports for the instrument, each support being moveable independently.

8. Apparatus according to claim 1, in which the instrument is one adapted to perform at least one of aspiration, injection, cauterization and electrical stimulation.

9. A support for a surgical instrument, comprising:
an interface for attaching the support at a location a guide for receiving a surgical instrument;
a data input for receiving location data relating to the guide;
a drive for adjusting the spatial location of the guide relative to the interface.

10. A support according to claim 9 comprising two such guides, and two such drives, each drive being arranged to adjust the location of one of the guides.

11. A support according to claim 9 in which the or each drive comprises at least a pair of motors, thereby to adjust the location of the associated guide in two dimensions.

12. A support according to claim 11, comprising a further motor to drive the or each guide in a third dimension transverse to each of the two dimensions.

13. A support according to claim 9 in which the location data is an error signal representing a difference between the actual location of the or each guide and a desired location.

14. A method of guiding an instrument to a location relative to a feature within a physiology, comprising the steps of:
performing a scan of the physiology to determine the location of the feature within the physiology relative to at least one external feature associated with the physiology;
providing an adjustable articulated arm having a tip and adapted to report a location of the tip;
employing the arm to determine a location of the or each external feature;
employing the arm to determine a location of the instrument;
with the thus acquired knowledge of the location of the instrument relative to the location of the feature, guiding the instrument to the feature.

15. A method according to claim 14 in which the scan is one of a computed tomography scan and a magnetic resonance imaging scan.

16. A method according to claim 14 in which the external feature is a marker attached to the physiology.

17. A method according to claim 14 in which the instrument is located on a guide adapted to constrain movement of the instrument.

18. A method according to claim 17 in which the instrument is supported by the guide.

19. A method according to claim 17 in which the instrument is elongate and the guide constrains translational movement of the instrument to translation along its elongate axis.

20. A method according to claim 17 in which the guide is moveable.

21. A method according to claim 20 in which movement of the guide is controlled servomechanically.

22. A method according to claim 17 in which the guide comprises at least two supports for the instrument, each support being moveable independently.

23. A method according to claim 17, in which there are at least three external features.

24. A method according to claim 14 in which the physiology is a human anatomy.

25. A method according to claim 14 in which the instrument, once guided to the location, is used to perform at least one of aspiration, injection, cauterization and electrical stimulation.
